(19) United States
(12) Patent Application Publication
(10) Pub. No.: US 2024/0174532 A1
(43) Pub. Date: May 30, 2024

(54) ELECTROLYZED WATER SPRAYING DEVICE

(71) Applicant: Panasonic Intellectual Property Management Co., Ltd., Kadoma-shi, Osaka (JP)

(72) Inventors: Fumiaki ASAI, Aichi (JP); Mai ISHIGURO, Aichi (JP)

(21) Appl. No.: 18/551,708

(22) PCT Filed: Jan. 24, 2022
(86) PCT No.: PCT/JP2022/002323

§ 371 Date: Sep. 21, 2023

(30) Foreign Application Priority Data
Mar. 31, 2021 (JP) 2021-059182

(51) Int. Cl. C02F 1/461; A61L 9/14

(52) U.S. Cl. CPC C02F 1/46104; A61L 9/145; A61L 2209/111; A61L 2209/134; A61L 2209/213; C02F 2201/46125; C02F 2201/46145; C02F 2201/4618; C02F 2209/42

(57) ABSTRACT

Electrolyzed water spraying device (1000) of the present disclosure includes water storage tank (100), electrolysis tank (200), first feeder (128), electrode part (210), humidification tank (300), second feeder (138), third feeder (228), sprayer (310), and controller (500). In a water shortage state in which water is insufficient in electrolysis tank (200), when water and electrolysis accelerator are fed to electrolysis tank (200), controller (500) executes initial processing of generating electrolyzed water having a second concentration lower than a first concentration and feeding the electrolyzed water having the second concentration to humidification tank (300) by third feeder (228), and executes, after the initial processing, normal processing of generating electrolyzed water having the first concentration and feeding the electrolyzed water having the first concentration to humidification tank (300) by third feeder (228).

### TECHNICAL FIELD

The present disclosure relates to an electrolyzed water spraying device that generates and sprays electrolyzed water.

### BACKGROUND ART

An electrolyzed water spraying device that generates and sprays electrolyzed water containing hypochlorous acid by electrolysis in order to remove (including inactivation of) bacteria, fungi, viruses, odors, and the like in the air is known (see, for example, Patent Literature 1). In order to generate hypochlorous acid, it is necessary to charge an electrolysis acceleration tablet such as a salt into water to be electrolyzed to generate water containing chloride ions in advance.

### CITATION LIST

### Patent Literature

PTL 1: Unexamined Japanese Patent Publication No. 2019-24811

### SUMMARY OF THE INVENTION

In the electrolyzed water spraying device, electrolyzed water is generated by executing electrolysis with electrodes provided in a water storage, and electrolyzed water stored in the water storage is brought into gas-liquid contact with air and sprayed. In such a structure, when the electrolyzed water stored in the water storage unit is contaminated by gas-liquid contact, the electrodes are likely to be deteriorated.

An object of the present disclosure is to provide a technique for preventing electrodes from deteriorating.

An electrolyzed water spraying device of the present disclosure includes: a water storage tank that stores water; an electrolysis tank configured to generate electrolyzed water from water to which an electrolysis accelerator has been charged; a first feeder configured to feed water from the water storage tank to the electrolysis tank; an electrode part that generates electrolyzed water in the electrolysis tank; a humidification tank that mixes the water fed from the water storage tank with the electrolyzed water fed from the electrolysis tank; a second feeder configured to feed water from the water storage tank to the humidification tank; a third feeder configured to feed the electrolyzed water from the electrolysis tank to the humidification tank; a sprayer that brings the electrolyzed water in the humidification tank into contact with air sucked from an intake port and sprays the electrolyzed water from a blow-out port; and a controller that controls the electrode part, the sprayer, the first feeder, the second feeder, and the third feeder. In a water shortage state in which water is insufficient in the electrolysis tank, when water and an electrolysis accelerator are fed to the electrolysis tank, the controller performs initial processing of generating electrolyzed water having a second concentration lower than a first concentration and feeding the electrolyzed water having the second concentration to the humidification tank by the third feeder, and performs, after the initial processing, normal processing of generating electrolyzed water having the first concentration and feeding the electrolyzed water having the first concentration to the humidification tank by the third feeder.

Any combinations of the above-described components and modifications of the expressions of the present disclosure among methods, devices, systems, recording media, and computer programs are also effective as aspects of the present disclosure.

According to the present disclosure, deterioration of electrodes can be prevented.

### BRIEF DESCRIPTION OF THE DRAWINGS

FIG. 1 is a diagram illustrating an internal configuration of an electrolyzed water spraying device according to an exemplary embodiment.

FIG. 2A is a diagram illustrating an outline of operations of the electrolyzed water spraying device of FIG. 1.

FIG. 2B is a diagram illustrating an outline of operations of the electrolyzed water spraying device of FIG. 1.

FIG. 2C is a diagram illustrating an outline of operations of the electrolyzed water spraying device of FIG. 1.

FIG. 3A is a diagram illustrating an outline of operations of the electrolyzed water spraying device of FIG. 1.

FIG. 3B is a diagram illustrating an outline of operations of the electrolyzed water spraying device of FIG. 1.

FIG. 4A is a diagram illustrating an outline of operations of the electrolyzed water spraying device of FIG. 1.

FIG. 4B is a diagram illustrating an outline of operations of the electrolyzed water spraying device of FIG. 1.

FIG. 5 is a flowchart illustrating a control procedure performed by the electrolyzed water spraying device of FIG. 1.

FIG. 6 is a flowchart illustrating a control procedure performed by the electrolyzed water spraying device according to a modification.

### DESCRIPTION OF EMBODIMENT

Before specifically describing an exemplary embodiment of the present disclosure, an outline of the exemplary embodiment will be explained. The present exemplary embodiment is an electrolyzed water spraying device that generates electrolyzed water based on water and an electrolysis accelerator and then sprays the electrolyzed water. A conventional electrolyzed water spraying device generates water containing chloride ions by dissolving an electrolysis accelerator in water in a water storage, and then generates electrolyzed water containing active oxygen species by electrolyzing the water containing chloride ions through energization to electrodes. In the electrolyzed water spraying device, the generated electrolyzed water and the air sucked from the outside are continuously brought into contact with each other in the water storage, and then the electrolyzed water is sprayed to the outside by the rotation of the fan. Therefore, the electrolyzed water in the water storage is easily contaminated by contact with air. When the electrolyzed water is contaminated, the electrodes are likely to be deteriorated.

In order to prevent the electrode from deteriorating, in the electrolyzed water spraying device according to the present exemplary embodiment, the water storage is divided into two water tanks, which are an electrolysis tank and a humidification tank. The electrolysis tank is provided with electrodes, and the electrode electrolyzes water containing chloride ions to generate electrolyzed water in the electrolysis tank. The electrolyzed water generated in the electrolysis tank is fed to the humidification tank. Furthermore, in the humidification tank, the electrolyzed water fed from the electrolysis tank is continuously brought into contact with air sucked from the outside, and then the electrolyzed water is sprayed to the outside by rotation of the fan. With such a configuration, the electrolyzed water in the electrolysis tank does not come into contact with air, making the electrolyzed water less likely to be contaminated, and the electrode is prevented from deteriorating.

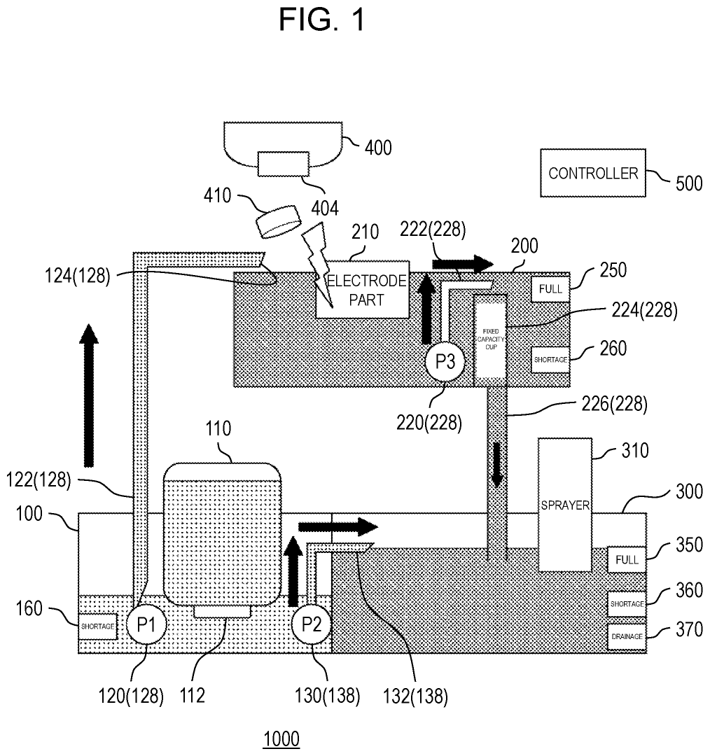

Hereinafter, electrolyzed water spraying device 1000 according to an exemplary embodiment of the present disclosure will be described with reference to the accompanying drawings. FIG. 1 shows an internal configuration of electrolyzed water spraying device 1000.

Electrolyzed water spraying device 1000 includes water storage tank 100, water feed tank 110, lid 112, first pump 120, first water feed pipe 122, feed port 124, second pump 130, second water feed pipe 132, shortage float 160, electrolysis tank 200, electrode part 210, third pump 220, third water feed pipe 222, fixed capacity cup 224, fourth water feed pipe 226, full capacity float 250, shortage float 260, humidification tank 300, sprayer 310, full capacity float 350, shortage float 360, drainage float 370, electrolysis accelerator charge part 400, charge port 404, electrolysis accelerator 410, and controller 500. Here, first pump 120, first water feed pipe 122, and feed port 124 are included in first feeder 128, second pump 130 and second water feed pipe 132 are included in second feeder 138, and third pump 220, third water feed pipe 222, fixed capacity cup 224, and fourth water feed pipe 226 are included in third feeder 228.

Hereinafter, (1) basic configuration, (2) initial processing, (3) normal processing, and (4) regeneration processing will be described in this order.

### (1) Basic Configuration

Water storage tank 100 has a box shape with a top surface opened, has a structure that can store water, and stores water fed from water feed tank 110 described later. For example, water storage tank 100 is disposed in a lower portion of electrolyzed water spraying device 1000. Water feed tank 110 is a tank that stores water in the inside thereof, and is detachable from water storage tank 100. Lid 112 is provided at an opening (not illustrated) of water feed tank 110, and an opening and closing portion (not illustrated) is provided at a center of lid 112. When the opening and closing portion is opened, water in water feed tank 110 is fed to water storage tank 100.

Specifically, when water feed tank 110 is attached to water storage tank 100 with the opening of water feed tank 110 facing downward, the opening and closing portion opens. That is, when water feed tank 110 containing water is attached to water storage tank 100, the opening and closing portion is opened to feed water to water storage tank 100, and water is stored in water storage tank 100. When the water level in water storage tank 100 rises and reaches lid 112, the opening of water feed tank 110 is sealed by water. Accordingly, the water feeding is stopped. When water remains in water feed tank 110, water in water feed tank 110 is fed to water storage tank 100 every time a water level in water storage tank 100 drops. As a result, the water level in water storage tank 100 is kept constant.

First pump 120 is disposed in water storage tank 100, and is connected to first water feed pipe 122. When operating in accordance with an instruction from controller 500, first pump 120 pumps up water stored in water storage tank 100 toward first water feed pipe 122. First water feed pipe 122 is a pipe connecting water storage tank 100 and electrolysis tank 200, and has a feed port 124 at an end on electrolysis tank 200 side. Water pumped up by first pump 120 flows in first water feed pipe 122, and is fed from feed port 124 to electrolysis tank 200. That is, first pump 120, first water feed pipe 122, and feed port 124 feed water from water storage tank 100 to electrolysis tank 200.

Second pump 130 is disposed in water storage tank 100, and is connected to second water feed pipe 132. When operating in accordance with an instruction from controller 500, second pump 130 pumps up water stored in water storage tank 100 toward second water feed pipe 132. Second water feed pipe 132 is a pipe connecting water storage tank 100 and humidification tank 300. Water pumped up by second pump 130 flows in second water feed pipe 132, and is fed to humidification tank 300. That is, second pump 130 and second water feed pipe 132 feed water from water storage tank 100 to humidification tank 300.

Electrolysis tank 200 has a box shape with a top surface opened, and is disposed below feed port 124. Electrolysis tank 200 stores water fed from feed port 124. On the upper side of electrolysis tank 200, electrolysis accelerator charge part 400 is disposed side by side with feed port 124. Electrolysis accelerator charge part 400 can be loaded with electrolysis accelerator 410 therein, and rotates a tablet charge member (not illustrated) when receiving an instruction to charge electrolysis accelerator 410 from controller 500. When the tablet charge member rotates, electrolysis accelerator 410 drops into electrolysis tank 200. Electrolysis accelerator charge part 400 counts the number of electrolysis accelerators 410 dropped into electrolysis tank 200, and stops the rotation of the tablet charge member when determining that one tablet of electrolysis accelerator 410 has dropped into electrolysis tank 200. That is, electrolysis accelerator charge part 400 charges electrolysis accelerator 410 into electrolysis tank 200. When electrolysis accelerator 410 dissolves into water in electrolysis tank 200, water containing chloride ions is generated in electrolysis tank 200. One example of electrolysis accelerator 410 is sodium chloride and is formed as an electrolysis acceleration tablet.

Electrode part 210 is installed in a manner to be immersed in water in electrolysis tank 200. By being applied with energization, electrode part 210 electrochemically electrolyzed water containing chloride ions in electrolysis tank 200 to generate electrolyzed water containing active oxygen species. Here, the active oxygen species are oxygen molecules having an oxidation activity higher than the oxidation activity of normal oxygen and related substances thereof. For example, active oxygen species include so-called narrow sense of active oxygen such as superoxide anion, singlet oxygen, hydroxyl radical, or hydrogen peroxide, and so-called broad sense of active oxygen such as ozone or hypochlorous acid (hypohalous acid).

Electrode part 210 generates electrolyzed water by repeating one cycle a plurality of times, where the one cycle is an energization time for performing energization for electrolysis and a time after stop of the energization, that is, a non-energization time, which is a time when the energization is not performed. By providing the non-energization time for electrode part 210, the life of electrode part 210 is extended. When the energization time is made longer than the non-energization time, electrolyzed water containing a larger amount of active oxygen species per cycle is generated. When the non-energization time is made longer than the energization time, generation of active oxygen species per cycle can be suppressed. Furthermore, when the amount of power during the energization time is increased, electrolyzed water containing a larger amount of active oxygen species is generated. As described above, it can be said that electrolysis tank 200 is a tank for generating the electrolyzed water from the water into which electrolysis accelerator 410 is charged.

Third pump 220 is disposed in electrolysis tank 200, and is connected to third water feed pipe 222. When operating in accordance with an instruction from controller 500, third pump 220 pumps up electrolyzed water stored in electrolysis tank 200 toward third water feed pipe 222. Third water feed pipe 222 is connected to fixed capacity cup 224, and feeds the electrolyzed water in electrolysis tank 200 to fixed capacity cup 224. Fixed capacity cup 224 is a square vessel having a fixed capacity, and stores a fixed amount of the electrolyzed water fed from third water feed pipe 222. Fixed capacity cup 224 is connected to fourth water feed pipe 226, and fourth water feed pipe 226 extends toward humidification tank 300. Electrolyzed water stored in fixed capacity cup 224 flows in fourth water feed pipe 226 and is fed to humidification tank 300. That is, third pump 220, third water feed pipe 222, fixed capacity cup 224, and fourth water feed pipe 226 feed the electrolyzed water from electrolysis tank 200 to humidification tank 300.

Humidification tank 300 has a box shape with a top surface opened, and mixes the water fed from water storage tank 100 and the electrolyzed water fed from electrolysis tank 200. This corresponds to diluting the electrolyzed water fed from electrolysis tank 200 with the water fed from water storage tank 100. Humidification tank 300 is provided with sprayer 310.

Sprayer 310 includes a fan (not illustrated) and a filter (filter). The fan is, for example, a sirocco fan, and rotates under the control of controller 500. When the fan rotates, air is sucked into electrolyzed water spraying device 1000 from an intake port (not illustrated) provided in a housing (not illustrated) of electrolyzed water spraying device 1000.

The filter is a member that brings the electrolyzed water stored in humidification tank 300 into contact with the indoor air flowing into electrolyzed water spraying device 1000 by the fan. The filter is formed in a cylindrical shape, and has a hole through which air can flow in a circumferential portion. The filter is rotatably incorporated in humidification tank 300 with a central axis as a rotation center so that one end of the filter is immersed in the electrolyzed water stored in humidification tank 300 to retain water. The filter is rotated by a drive unit (not illustrated) to bring the electrolyzed water and the indoor air into continuous contact with each other.

An air path leading from the intake port to the filter, the fan, and the blow-out port (not illustrated) is formed. When the fan rotates, the external air sucked from the intake port and entering the air path is blown out of electrolyzed water spraying device 1000 sequentially through the filter, the fan, and the blow-out port. Thus, the electrolyzed water in humidification tank 300 is sprayed to the outside. Electrolyzed water spraying device 1000 does not necessarily spray electrolyzed water itself, and spraying of the active oxygen species resultantly derived from (including volatilization of) electrolyzed water is also included in the electrolyzed water spraying.

Shortage float 160 provided in water storage tank 100, full capacity float 250 and shortage float 260 provided in electrolysis tank 200, and full capacity float 350, shortage float 360, and drainage float 370 provided in humidification tank 300 each detects whether or not water or electrolyzed water is present. Here, water and electrolyzed water may be collectively referred to as “water”. Shortage float 160, full capacity float 250, shortage float 260, full capacity float 350, shortage float 360, and drainage float 370 are collectively referred to as “floats”. Each of the floats has buoyancy and a magnet (not illustrated), and the position of the magnet is detected by a detection portion (not illustrated). In a case where water is present up to the position of the float, the float moves to a predetermined position by buoyancy, and the detection portion detects the magnet provided on the float portion. On the other hand, in a case where water is not present up to the position of the float, the detection portion cannot detect the magnet provided on the float.

Shortage float 160 detects water shortage of water storage tank 100, full capacity float 250 detects the full capacity of electrolysis tank 200, and shortage float 260 detects water shortage of electrolysis tank 200. Here, the water shortage does not have to be a 100% water shortage, and a slight amount of water may remain. In the present example, shortage float 260 may be referred to as a water shortage detector. In addition, full capacity float 350 detects the full capacity of humidification tank 300, shortage float 360 detects the water shortage of humidification tank 300, and drainage float 370 detects the drainage level of humidification tank 300. Here, the full capacity does not have to be 100% full capacity, and may be a water level at which water can be further fed. Each float transmits a detection result to controller 500.

Controller 500 receives detection results from shortage float 160, full capacity float 250, shortage float 260, full capacity float 350, shortage float 360, and drainage float 370. Controller 500 controls electrode part 210, sprayer 310, electrolysis accelerator charge part 400, first feeder 128, second feeder 138, and third feeder 228. Details of the processing of controller 500 will be described later.

As an example, the concentration of the electrolyzed water to be generated in electrolysis tank 200 is a concentration (hereinafter, referred to as “first concentration”) within a range from 30 ppm to 200 ppm. The concentration of the electrolyzed water to be diluted in humidification tank 300 is within a range from 3 ppm to 50 ppm. The concentration of the electrolyzed water to be diluted in humidification tank 300 is set to be lower than the concentration of the electrolyzed water to be generated in electrolysis tank 200.

### (2) Initial Processing

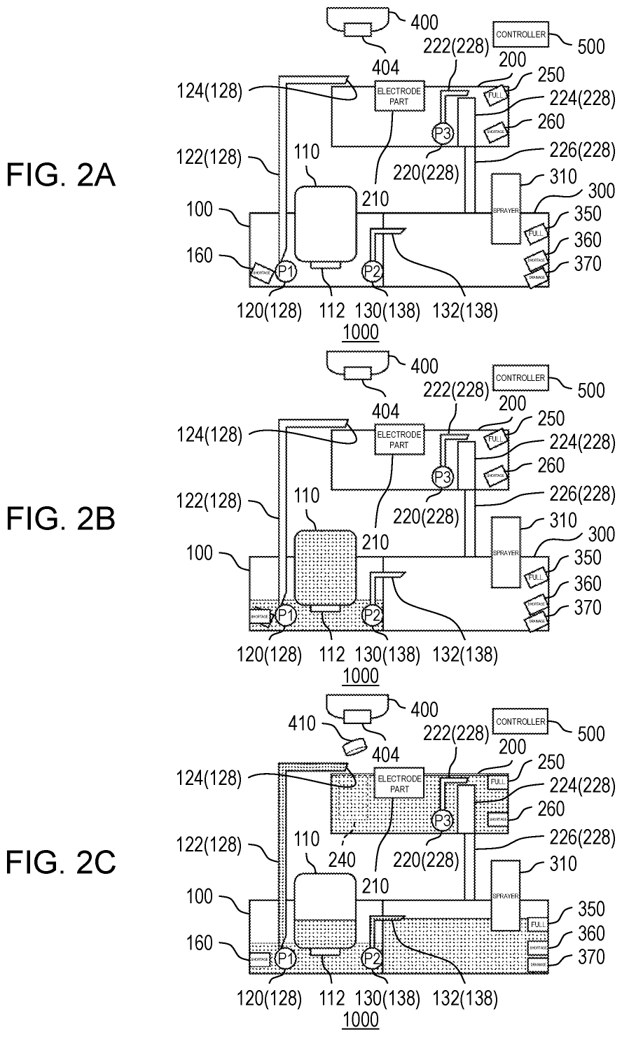

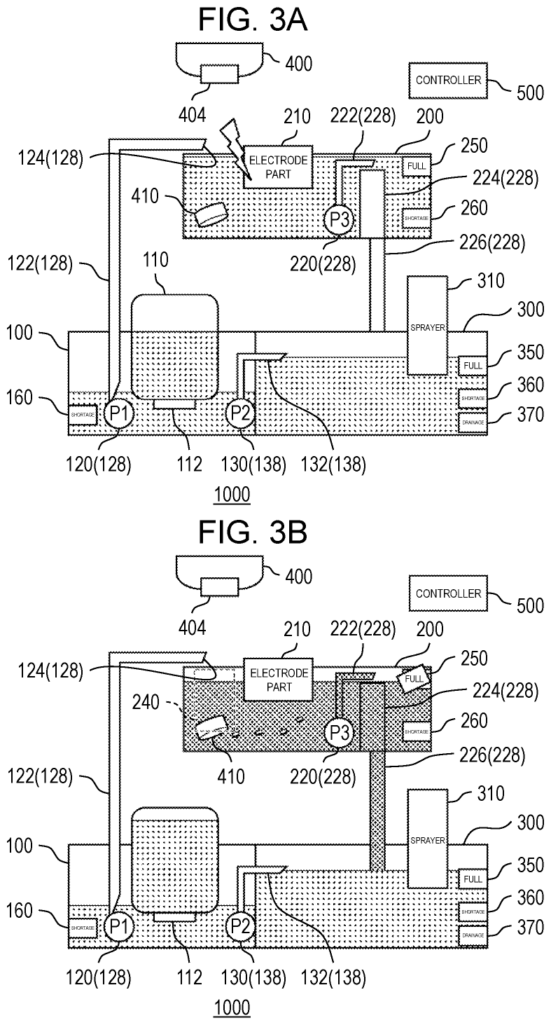

The initial processing is processing from a state in which the water in water storage tank 100, electrolysis tank 200, and humidification tank 300 is in a water shortage state, specifically, a state in which there is no water, to a state in which the initial stage of spraying of the electrolyzed water is executed. In the following, FIGS. 2A to 2C, 3A, and 3B are also referred to describe the initial processing. FIGS. 2A to 2C each illustrates an outline of operations of electrolyzed water spraying device 1000.

FIG. 2A illustrates a water shortage state in which water is insufficient in water storage tank 100, electrolysis tank 200, and humidification tank 300. This corresponds to a case where electrolyzed water spraying device 1000 is installed after electrolyzed water spraying device 1000 is purchased. This also corresponds to the case after maintenance of water storage tank 100, electrolysis tank 200, and humidification tank 300.

FIG. 2B is a state following FIG. 2A. The user charges water into water feed tank 110, and installs water feed tank 110 to water storage tank 100. When water feed tank 110 is attached to water storage tank 100, water is fed from water feed tank 110 to water storage tank 100 by opening the opening and closing portion of lid 112.

FIG. 2C is a state following FIG. 2B. Controller 500 operates second pump 130 to feed the water in water storage tank 100 to humidification tank 300. The water is fed until full capacity float 350 detects a full capacity of the water. As a result, humidification tank 300 stores water in a state of being a full capacity.

Controller 500 operates first pump 120 to feed water in water storage tank 100 to electrolysis tank 200. At that time, water is fed for a certain period of time within which electrolysis tank 200 is not filled to a full capacity. As a result of the water feeding, the water surface of electrolysis tank 200 is at a water level lower than the water level of a full capacity. Feed region 240 is disposed on a part of the water surface of electrolysis tank 200, and feed region 240 is located below feed port 124 and charge port 404. After the water feeding has been completed, controller 500 causes electrolysis accelerator 410 to be dropped from charge port 404 toward feed region 240 of electrolysis tank 200. As a result, electrolysis accelerator 410 is present in feed region 240 and begins to dissolve in water.

Subsequently, controller 500 operates first pump 120 again to feed water in water storage tank 100 to electrolysis tank 200. At this time, since water is fed from feed port 124 toward feed region 240, the dissolution of electrolysis accelerator 410 further proceeds by the pressure of the fed water. The water is fed until full capacity float 250 detects a full capacity of the water. As a result, humidification tank 300 stores water containing chloride ions in which a part of electrolysis accelerator 410 is dissolved in a state of being a full capacity.

FIGS. 3A and 3B each illustrates an outline of operations of electrolyzed water spraying device 1000 following FIGS. 2A to 2C.

FIG. 3A is a state following FIG. 2C. Controller 500 executes energization to electrode part 210 to electrolyze water containing chloride ions to generate electrolyzed water. Here, the electrolysis time is set to a time (for example, ten minutes) shorter than a time (for example, forty minutes) required for generating the electrolyzed water having the first concentration. As a result, electrolyzed water having a second concentration lower than the first concentration is generated.

FIG. 3B is a state following FIG. 3A. When the electrolyzed water having the second concentration is generated, controller 500 operates third pump 220 to feed the electrolyzed water having the second concentration to humidification tank 300. At this time, since fixed capacity cup 224 is used, the electrolyzed water having the second concentration corresponding to the capacity of fixed capacity cup 224 is fed to humidification tank 300. Electrolyzed water having the second concentration is diluted in humidification tank 300. After stopping third pump 220, controller 500 operates sprayer 310 to spray the electrolyzed water in humidification tank 300 to the outside of electrolyzed water spraying device 1000. That is, the spraying of the electrolyzed water is started from a time shorter than forty minutes.

### (3) Normal Processing

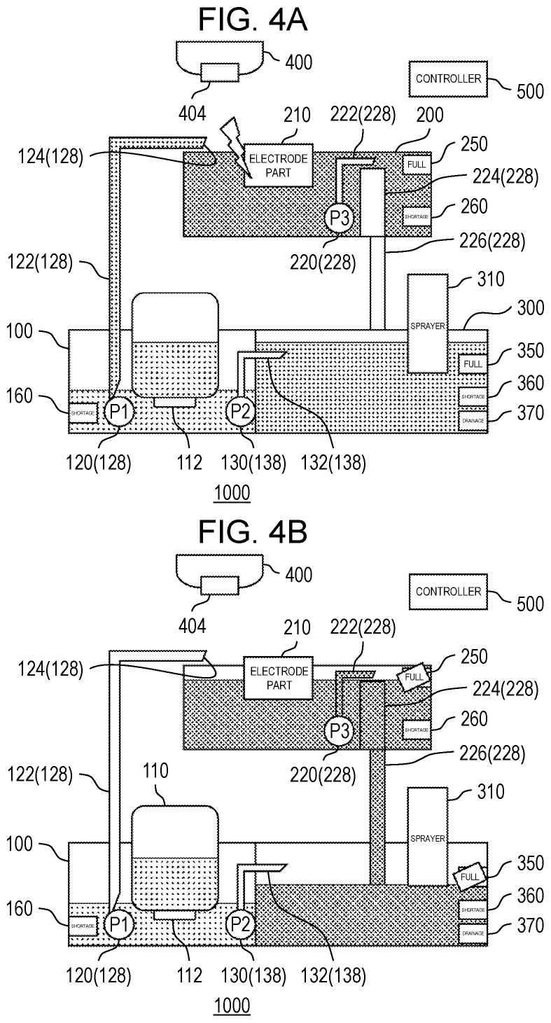

The normal processing is processing for spraying electrolyzed water having a desired concentration. FIGS. 4A and 4B each illustrates an outline of operations of electrolyzed water spraying device 1000 following FIGS. 3A and 3B.

FIG. 4A is a state following FIG. 3B. Since a part of the electrolyzed water having the second concentration in electrolysis tank 200 is fed to humidification tank 300, the electrolyzed water having the second concentration is stored in electrolysis tank 200 in a state of not being a full capacity. Controller 500 operates first pump 120 to feed water in water storage tank 100 to electrolysis tank 200. At this time, since water is fed from feed port 124 toward feed region 240, the remaining electrolysis accelerator 410 is further dissolved by the pressure of the fed water. The water is fed until full capacity float 250 detects a full capacity of the water. As a result, humidification tank 300 becomes in a state of being a full capacity of the water. After the water feeding to electrolysis tank 200 has been completed, controller 500 executes energization to electrode part 210 to generate electrolyzed water by electrolysis. Here, the electrolysis time is a time required for generating the electrolyzed water having the first concentration (for example, forty minutes). As a result, electrolyzed water having the first concentration is generated.

FIG. 4B is a state following FIG. 4A. When the electrolyzed water having the first concentration is generated, controller 500 operates third pump 220 to feed the electrolyzed water having the first concentration to humidification tank 300. At this time, since fixed capacity cup 224 is used, the electrolyzed water having the first concentration corresponding to the capacity of fixed capacity cup 224 is fed to the humidification tank 300. Electrolyzed water having the first concentration is diluted in humidification tank 300. After stopping third pump 220, controller 500 operates sprayer 310 to spray the electrolyzed water in humidification tank 300 to the outside of electrolyzed water spraying device 1000.

When the electrolyzed water is sprayed, the amount of the electrolyzed water in humidification tank 300 decreases. When shortage float 360 detects the water shortage, the controller 500 operates third pump 220 to feed the electrolyzed water having the first concentration to humidification tank 300 by the capacity of fixed capacity cup 224, and operates second pump 130 to feed the water in water storage tank 100 to humidification tank 300 to a full capacity. Thus, the spraying of the electrolyzed water is continued. Such processing is repeated until shortage float 260 detects water shortage.

### (4) Regeneration Processing

The regeneration processing is processing for executing the normal processing again when shortage float 260 detects water shortage, that is, when the electrolyzed water in electrolysis tank 200 becomes short of water. When a shortage of water is detected by shortage float 260 after feeding the electrolyzed water having the first concentration to humidification tank 300, controller 500 starts feeding water to electrolysis tank 200 by first feeder 128. That is, controller 500 does not feed water to electrolysis tank 200 until electrolysis tank 200 becomes short of water. This is because the concentration of the electrolyzed water in electrolysis tank 200 is maintained at the first concentration by not feeding water. This is also because impurities such as inorganic salt compounds are less likely to remain in electrolysis tank 200 by making old electrolyzed water less likely to remain in electrolysis tank 200. This reduces the frequency of maintenance of electrolysis tank 200.

Here, same as the initial processing, controller 500 executes water feeding for a certain period of time within which electrolysis tank 200 is not filled to a full capacity of the water. Subsequently, controller 500 causes electrolysis accelerator 410 to be dropped from charge port 404 toward feed region 240 of electrolysis tank 200, and executes water feeding to electrolysis tank 200 to a full capacity. In addition, controller 500 executes energization to electrode part 210 to generate the electrolyzed water having the second concentration, and then feeds the electrolyzed water having the second concentration from electrolysis tank 200 to humidification tank 300. That is, the same processing as a part of the initial processing is executed. Subsequently, normal processing is executed.

### (Modifications)

When executing the regeneration processing, unlike when executing the initial processing, the electrolyzed water is present in humidification tank 300. Therefore, the processing of generating the electrolyzed water having the second concentration and feeding the electrolyzed water having the second concentration from electrolysis tank 200 to humidification tank 300 may be omitted. Such a modification will be described below.

Controller 500 executes water feeding for a certain period of time within which electrolysis tank 200 is not filled to a full capacity of the water. Subsequently, controller 500 causes electrolysis accelerator 410 to be dropped from charge port 404 toward feed region 240 of electrolysis tank 200. Subsequently, controller 500 waits for a certain period of time. The waiting period may be shorter or longer than ten minutes, i.e., the electrolysis time in the initial processing. Thereafter, controller 500 executes feeding water to a full capacity of electrolysis tank 200. Subsequently, normal processing is executed. That is, the electrolyzed water having the second concentration is not generated by the electrolysis, but the electrolyzed water having the first concentration is generated.

The subject of devices, systems, or methods in the present disclosure includes a computer. This computer executes the program, thereby implementing the function of the subject of devices, systems, or methods in the present disclosure. The computer includes, as a main hardware configuration, a processor that acts according to a program. The type of the processor is not limited as long as the processor can implement the function by executing the program. The processor includes one or a plurality of electronic circuits including a semiconductor integrated circuit (IC) or a large scale integration (LSI). The plurality of electronic circuits may be integrated on one chip or may be provided on a plurality of chips. The plurality of chips may be aggregated into one device or may be provided in a plurality of devices. The program is recorded in a computer-readable non-transitory recording medium such as a read-only memory (ROM), an optical disk, or a hard disk drive. The program may be stored in advance in a recording medium, or may be fed to the recording medium via a wide area communication network including the Internet.

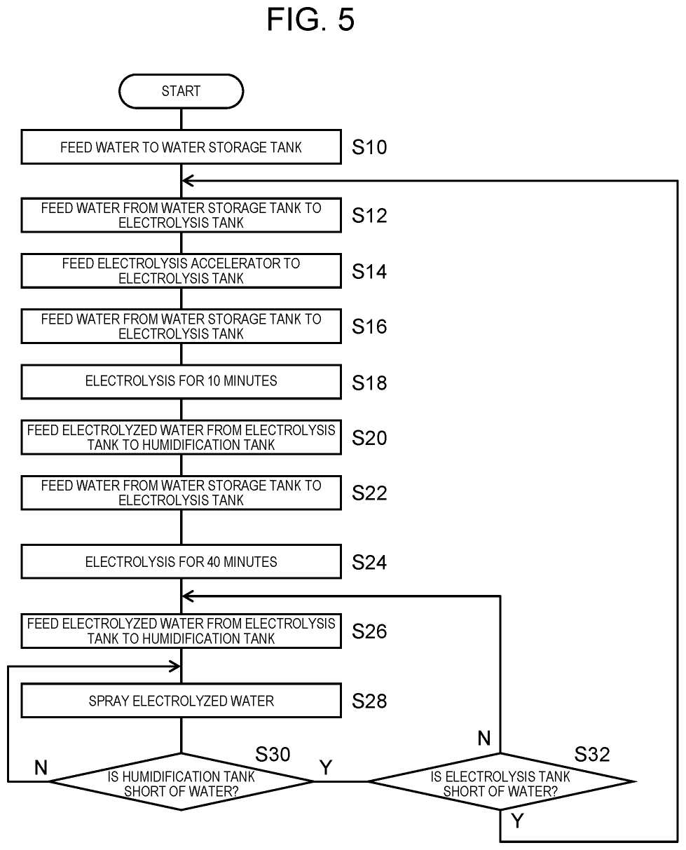

Operations of electrolyzed water spraying device 1000 having the above configuration will be described. FIG. 5 is a flowchart illustrating a control procedure performed by electrolyzed water spraying device 1000. Note that the following control may be executed by controller 500.

First, water is fed to water storage tank 100 (S10).

Next, water is fed from water storage tank 100 to electrolysis tank 200 by an amount smaller than a full capacity (S12).

Next, electrolysis accelerator 410 is fed to electrolysis tank 200 (S14).

Next, water is fed from water storage tank 100 to electrolysis tank 200 to a full capacity (S16).

Next, electrode part 210 executes electrolysis for ten minutes (S18).

Next, electrolyzed water having the second concentration is fed from electrolysis tank 200 to humidification tank 300 (S20). At this time, the electrolyzed water is sprayed in humidification tank 300.

Next, water is fed from water storage tank 100 to electrolysis tank 200 to a full capacity (S22).

Next, electrode part 210 executes electrolysis for forty minutes (S24).

Next, electrolyzed water having the first concentration is fed from electrolysis tank 200 to humidification tank 300 (S26).

Next, sprayer 310 sprays the electrolyzed water (S28).

Next, it is determined whether the water shortage of humidification tank 300 is detected by shortage float 360 (S30). When it is determined that humidification tank 300 is not short of water (N in S30), the processing returns to step S28. On the other hand, when it is determined that humidification tank 300 is short of water (Y in S30), it is determined whether the water shortage in electrolysis tank 200 is detected by shortage float 260 (S32). When it is determined that electrolysis tank 200 is not short of water (N in S32), the processing returns to step S26. On the other hand, when it is determined that electrolysis tank 200 is short of water (Y in S32), the processing returns to step S12.

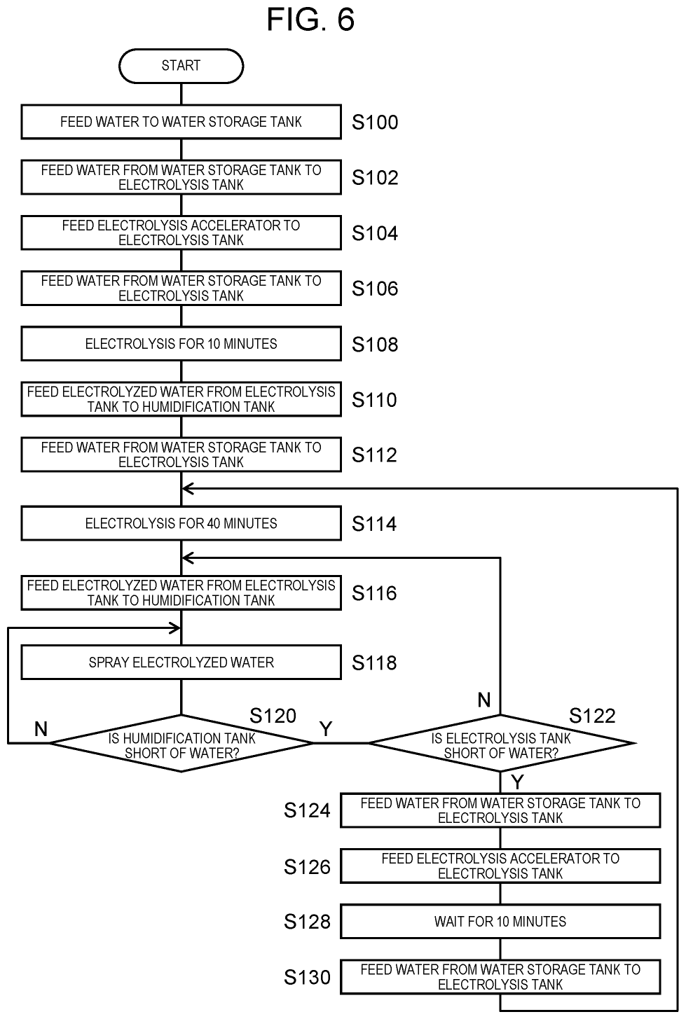

FIG. 6 is a flowchart illustrating a control procedure performed by the electrolyzed water spraying device 1000 according to a modification. Note that the following control may be executed by controller 500.

First, water is fed to water storage tank 100 (S100).

Next, water is fed from water storage tank 100 to electrolysis tank 200 by an amount smaller than a full capacity (S102).

Next, electrolysis accelerator 410 is fed to electrolysis tank 200 (S104).

Next, water is fed from water storage tank 100 to electrolysis tank 200 to a full capacity (S106).

Next, electrode part 210 executes electrolysis for ten minutes (S108).

Next, electrolyzed water having the second concentration is fed from electrolysis tank 200 to humidification tank 300 (S110). At this time, the electrolyzed water is sprayed in humidification tank 300.

Next, water is fed from water storage tank 100 to electrolysis tank 200 to a full capacity (S112).

Next, electrode part 210 executes electrolysis for forty minutes (S114).

Next, electrolyzed water having the first concentration is fed from electrolysis tank 200 to humidification tank 300 (S116).

Next, sprayer 310 sprays the electrolyzed water (S118).

Next, it is determined whether the water shortage of humidification tank 300 is detected by shortage float 360 (S120). When it is determined that humidification tank 300 is not short of water (N in S120), the processing returns to step S118. On the other hand, when it is determined that humidification tank 300 is short of water (Y in S120), it is determined whether the water shortage in electrolysis tank 200 is detected by shortage float 260 (S122). When it is determined that electrolysis tank 200 is not short of water (N in S122), the processing returns to step S116. On the other hand, when it is determined that the electrolysis tank 200 is short of water (Y in S122), water is fed from water storage tank 100 to electrolysis tank 200 by an amount smaller than the full capacity (S124). Thereafter, electrolysis accelerator 410 is fed to electrolysis tank 200 (S126), and the processing is waited for ten minutes (S128). Next, water is fed from water storage tank 100 to electrolysis tank 200 to a full capacity (S130) and the processing returns to step S114.

According to electrolyzed water spraying device 1000 of the present exemplary embodiment, since the water storage is divided into water storage tank 100, electrolysis tank 200, and humidification tank 300, occurrence of gas-liquid contact with water in electrolysis tank 200 used by electrode part 210 can be prevented. In addition, since the occurrence of gas-liquid contact with water in electrolysis tank 200 is prevented, the water in electrolysis tank 200 can be made less likely to be contaminated. In addition, since the water in electrolysis tank 200 is less likely to be contaminated, deterioration of electrodes can be prevented. In addition, since the electrolyzed water having the second concentration is fed to humidification tank 300 and sprayed, the period required until spraying the electrolyzed water can be shortened. In addition, since the electrolyzed water having the first concentration is generated following the electrolyzed water having the second concentration, the electrolyzed water having a desired concentration can be sprayed. In addition, since electrolysis accelerator 410 is charged toward feed region 240 and water is fed toward feed region 240, the dissolution of electrolysis accelerator 410 can be facilitated by the pressure of water. In addition, since the electrolyzed water having the first concentration is generated by the normal processing after the water is fed to electrolysis tank 200, electrolysis accelerator 410 can be easily dissolved.

In addition, when the water shortage is detected, water is fed to electrolysis tank 200 by first feeder 128, making the water feeding unnecessary until the water shortage is detected. In addition, since the water feeding becomes unnecessary until the water shortage is detected, the concentration of the electrolyzed water in electrolysis tank 200 can be maintained. In addition, since the water feeding becomes unnecessary until the water shortage is detected, the impurities remaining in electrolysis tank 200 can be flowed. Since a part of the initial processing is executed as the regeneration processing, the operation can be simplified. Since the electrolyzed water having the second concentration is not generated during the regeneration processing, the electrolyzed water having the first concentration can be efficiently generated.

An outline of an aspect of the present disclosure is as follows. Electrolyzed water spraying device (1000) of the present disclosure includes: water storage tank (100) that stores water; electrolysis tank (200) that generates electrolyzed water from water to which electrolysis accelerator (410) has been charged; first feeder (128) configured to feed water from water storage tank (100) to electrolysis tank (200); electrode part (210) that generates electrolyzed water in electrolysis tank (200); humidification tank (300) that mixes the water fed from water storage tank (100) with the electrolyzed water fed from electrolysis tank (200); second feeder (138) configured to feed water from water storage tank (100) to humidification tank (300); third feeder (228) configured to feed the electrolyzed water from electrolysis tank (200) to humidification tank (300); sprayer (310) that brings the electrolyzed water in humidification tank (300) into contact with air sucked from an intake port and sprays the electrolyzed water from a blow-out port; and controller (500) that controls electrode part (210), sprayer (310), first feeder (128), second feeder (138), and third feeder (228).

In a water shortage state in which water is insufficient in electrolysis tank (200), when water and electrolysis accelerator (410) are fed to electrolysis tank (200), controller (500) performs initial processing of generating electrolyzed water having a second concentration lower than a first concentration and feeding the electrolyzed water having the second concentration to humidification tank (300) by third feeder (228), and performs, after the initial processing, normal processing of generating electrolyzed water having the first concentration and feeding the electrolyzed water having the first concentration to humidification tank (300) by third feeder (228).

Electrolyzed water spraying device (1000) of the present disclosure may further includes electrolysis accelerator charge part (400) that charges electrolysis accelerator (410) into electrolysis tank (200). First feeder (128) may feed water from water storage tank (100) to electrolysis tank (200) toward feed region (240) configuring a part of a water surface of electrolysis tank (200), and electrolysis accelerator charge part (400) may charge electrolysis accelerator (410) toward feed region (240).

Furthermore, after executing the initial processing, controller (500) of the present disclosure may, after feeding water to electrolysis tank (200) by first feeder (128), generate electrolyzed water having the first concentration by the normal processing.

Electrolyzed water spraying device (1000) of the present disclosure may further include water shortage detector (260) that detects water shortage in electrolysis tank (200). When the water shortage is detected by water shortage detector (260) after feeding the electrolyzed water having the first concentration to humidification tank (300), controller (500) may feed water to electrolysis tank (200) by first feeder (128).

In addition, controller (500) of the present disclosure may execute regeneration processing of feeding water to electrolysis tank (200) by first feeder (128), feeding electrolysis accelerator (410) to electrolysis tank (200), then generating electrolyzed water having the second concentration, and feeding the electrolyzed water having the second concentration to humidification tank (300) by third feeder (228), and execute normal processing after the regeneration processing.

In addition, controller (500) of the present disclosure may execute regeneration processing of feeding a certain amount of the water to electrolysis tank (200) by first feeder (128), feeding electrolysis accelerator (410) to electrolysis tank (200), then waiting for a certain period of time, and feeding the water to electrolysis tank (200) by first feeder (128), and execute the normal processing after the regeneration processing.

The present disclosure has been described above based on the exemplary embodiment. It will be understood by those skilled in the art that the exemplary embodiment is merely an example, other modifications in which components and/or processes of the exemplary embodiment are variously combined are possible, and the other modifications still fall within the scope of the present disclosure.

In the exemplary embodiment, the shortage float 260 detects the water shortage by the position of the magnet in the float. However, the present disclosure is not limited thereto, and for example, the water shortage may be detected based on the number of times of feeding the electrolyzed water with fixed capacity cup 224. For example, when the electrolysis tank 200 is 1000 ml and the fixed capacity cup 224 is 250 ml, the water shortage is detected when the electrolyzed water is supplied four times with the fixed capacity cup 224. This modification can increase the degree of freedom in the configuration.

In the exemplary embodiment, water or electrolyzed water is fed when a shortage of water is detected. However, the present disclosure is not limited thereto, and for example, after feeding water or electrolyzed water, the next feeding may be performed after a certain period of time has elapsed. This modification can increase the degree of freedom in the configuration.

Furthermore, controller 500 may further include a storage configured to store a control content currently being executed. An example of the storage is a nonvolatile memory. Controller 500 periodically stores the control content currently being executed in the storage as necessary. When the power supply of electrolyzed water spraying device 1000 is shut off and then the power supply is restored, controller 500 resumes the control content that has been being executed and stored in the storage. For example, when the control content stored in the storage when the power supply of electrolyzed water spraying device 1000 is shut off is the content of electrolysis that is being executed for five minutes in step S18 of generating the electrolyzed water having the second concentration, when the power supply is restored thereafter, controller 500 performs control to execute electrolysis for the remaining five minutes necessary for generating the electrolyzed water having the second concentration. Furthermore, for example, when the control content stored in the storage when the power supply of electrolyzed water spraying device 1000 is shut off is step S28 of spraying the electrolyzed water, when the power supply is restored thereafter, controller 500 resumes the step of spraying the electrolyzed water. As a result, even when the power supply of electrolyzed water spraying device 1000 is shut off and then the power supply is restored, correct control contents can be performed.

### REFERENCE MARKS IN THE DRAWINGS

100 water storage tank
110 water feed tank
112 lid
120 first pump
122 first water feed pipe
124 feed port
128 first feeder
130 second pump
132 second water feed pipe
138 second feeder
160 shortage float
200 electrolysis tank
210 electrode part
220 third pump
222 third water feed pipe
224 fixed capacity cup
226 fourth water feed pipe
228 third feeder
240 feed region
250 full capacity float
260 shortage float
300 humidification tank
310 sprayer
350 full capacity float
360 shortage float
370 drainage float
400 electrolysis accelerator charge part
404 charge port
410 electrolysis accelerator
500 controller
1000 electrolyzed water spraying device

What is claimed is:

1. An electrolyzed water spraying device comprising:
a water storage tank that stores water;
an electrolysis tank configured to generate electrolyzed water from the water charged with an electrolysis accelerator;
a first feeder configured to feed the water from the water storage tank to the electrolysis tank;
an electrode part that generates the electrolyzed water in the electrolysis tank;
a humidification tank configured to mix the water fed from the water storage tank and the electrolyzed water fed from the electrolysis tank;
a second feeder configured to feed the water from the water storage tank to the humidification tank;
a third feeder configured to feed the electrolyzed water from the electrolysis tank to the humidification tank;
a sprayer configured to bring the electrolyzed water in the humidification tank into contact with air sucked from an intake port and sprays the electrolyzed water from a blow-out port; and
a controller configured to control the electrode part, the sprayer, the first feeder, the second feeder, and the third feeder,
wherein
the controller is configured to,
execute initial processing of generating the electrolyzed water having a second concentration lower than a first concentration and feeding the electrolyzed water having the second concentration to the humidification tank by the third feeder when the water and the electrolysis accelerator are fed to the electrolysis tank in a water shortage state where the water is insufficient in the electrolysis tank, and
execute, after the initial processing, normal processing of generating the electrolyzed water having the first concentration and feeding the electrolyzed water having the first concentration to the humidification tank by the third feeder.

2. The electrolyzed water spraying device according to claim 1, further comprising an electrolysis accelerator charge part configured to charge the electrolysis accelerator into the electrolysis tank, wherein
the first feeder is configured to feed the water from the water storage tank to the electrolysis tank toward a feed region configuring a part of a water surface of the electrolysis tank, and
the electrolysis accelerator charge part is configured to charge the electrolysis accelerator toward the feed region.

3. The electrolyzed water spraying device according to claim 1, wherein the controller is configured to, after executing the initial processing, feed the water to the electrolysis tank by the first feeder, then generate electrolyzed water having the first concentration by the normal processing.

4. The electrolyzed water spraying device according to claim 1, further comprising a water shortage detector configured to detect a water shortage in the electrolysis tank, wherein
when a water shortage is detected by the water shortage detector after feeding the electrolyzed water having the first concentration to the humidification tank, the controller is configured to feed the water to the electrolysis tank by the first feeder.

5. The electrolyzed water spraying device according to claim 4, wherein the controller is configured to
after the water shortage is detected by the water shortage detector, execute regeneration processing of feeding the water to the electrolysis tank by the first feeder, feeding the electrolysis accelerator to the electrolysis tank, then generating the electrolyzed water having the second concentration, and feeding the electrolyzed water having the second concentration to the humidification tank by the third feeder, and
execute the normal processing after the regeneration processing.

6. The electrolyzed water spraying device according to claim 4, wherein the controller is configured to
after the water shortage is detected by the water shortage detector, execute regeneration processing of feeding a certain amount of the water to the electrolysis tank by the first feeder, feeding the electrolysis accelerator to the electrolysis tank, then waiting for a certain period of time, and feeding the water to the electrolysis tank by the first feeder, and
execute the normal processing after the regeneration processing.

7. The electrolyzed water spraying device according to claim 1, wherein the controller
further includes a storage configured to store a control content currently being executed, and
when a power supply of the electrolyzed water spraying device is shut off and then the power supply is restored, resumes the control content that is currently being executed and stored in the storage.
